(19) United States
(12) Patent Application Publication
(10) Pub. No.: US 2014/0043488 A1
(43) Pub. Date: Feb. 13, 2014

(54) System and Method for Drug Detection Using SWIR

(71) Applicant: ChemImage Corporation, Pittsburgh, PA (US)

(72) Inventors: Patrick Treado, Pittsburgh, PA (US); Matthew Nelson, Harrison City, PA (US); Charles Gardner, Gibsonia, PA (US)

(73) Assignee: ChemImage Corporation, Pittsburgh, PA (US)

(21) Appl. No.: 14/055,509

(22) Filed: Oct. 16, 2013

Related U.S. Application Data
(63) Continuation-in-part of application No. 12/924,831, filed on Oct. 6, 2010.
(60) Provisional application No. 61/714,570, filed on Oct. 16, 2012.

(51) Int. Cl. G01N 33/15

(52) U.S. Cl. CPC G01N 33/15
USPC 348/164

(57) ABSTRACT

A method for detecting unknown materials, such as drugs. A first location is surveyed using a video capture device to identify a second location comprising an unknown material. The second location is interrogated using SWIR spectroscopic and/or imaging methods to generate a SWIR hyperspectral image. The SWIR hyperspectral image is analyzed to associate the unknown material with a known drug material. A system for detecting unknown materials, such as drugs comprising a first collection lens for collecting interacted photons from a first location and a visible imaging device for generating a visible image. A second collection lens may collect a plurality of interacted photons from a second location and a tunable filter may filter the interacted photons. A spectroscopic imaging device may detect the interacted photons and generate a SWIR hyperspectral image. A processor may analyze the SWIR hypespectral image to associate an unknown material with a known material.

### RELATED APPLICATIONS

This Application is a continuation-in-part to pending U.S. patent application Ser. No. 12/924,831, filed on Oct. 6, 2010, entitled “System and Methods for Explosive Detection using SWIR.” This Application also claims priority under 35 U.S.C. §119(e) to pending U.S. Provisional Patent Application No. 61/714,570, filed on Oct. 16, 2012, entitled “System and Method for Material Detection Using Short Wave Infrared Hyperspectral Imaging.” These applications are hereby incorporated by reference in their entireties.

### BACKGROUND

Spectroscopic imaging combines digital imaging and molecular spectroscopy techniques, which can include Raman scattering, fluorescence, photoluminescence, ultraviolet, visible and infrared absorption spectroscopies. When applied to the chemical analysis of materials, spectroscopic imaging is commonly referred to as chemical imaging. Instruments for performing spectroscopic (i.e. chemical) imaging typically comprise an illumination source, image gathering optics, focal plane array imaging detectors and imaging spectrometers.

In general, the sample size determines the choice of image gathering optic. For example, a microscope is typically employed for the analysis of sub micron to millimeter spatial dimension samples. For larger objects, in the range of millimeter to meter dimensions, macro lens optics are appropriate. For samples located within relatively inaccessible environments, flexible fiberscope or rigid borescopes can be employed. For very large scale objects, such as planetary objects, telescopes are appropriate image gathering optics.

For detection of images formed by the various optical systems, two-dimensional, imaging focal plane array (FPA) detectors are typically employed. The choice of FPA detector is governed by the spectroscopic technique employed to characterize the sample of interest. For example, silicon (Si) charge-coupled device (CCD) detectors or complementary metal-oxide semiconductor (CMOS) detectors are typically employed with visible wavelength fluorescence and Raman spectroscopic imaging systems, while indium gallium arsenide (InGaAs) FPA detectors are typically employed with near-infrared spectroscopic imaging systems.

Spectroscopic imaging of a sample can be implemented by one of two methods. First, a point-source illumination can be provided on the sample to measure the spectra at each point of the illuminated area. Second, spectra can be collected over the an entire area encompassing the sample simultaneously using an electronically tunable optical imaging filter such as an acousto-optic tunable filter (AOTF) or a liquid crystal tunable filter (LCTF). Here, the organic material in such optical filters is actively aligned by applied voltages to produce the desired bandpass and transmission function. The spectra obtained for each pixel of such an image thereby forms a complex data set referred to as a hyperspectral image which contains the intensity values at numerous wavelengths or the wavelength dependence of each pixel element in this image.

Spectroscopic devices operate over a range of wavelengths due to the operation ranges of the detectors or tunable filters possible. This enables analysis in the ultraviolet (UV), visible (VIS), near infrared (NIR), short-wave infrared (SWIR), mid infrared (MIR) wavelengths and to some overlapping ranges. These correspond to wavelengths of about 180-380 nm (UV), about 380-700 nm (VIS), about 700-2500 nm (NIR), about 850-1700 nm (SWIR), 700-1700 (VIS-NIR), about 2500-5000 nm (MIR), and about 5000-25000 nm (LWIR).

There exists a need for a system and method for detecting drug materials. It would be advantageous if the system and method could operate using either passive or active illumination and therefore enable both daytime and covert nighttime operation. It would also be advantageous if the system and method could operate in one or more configurations such as stationary and on-the-move (OTM).

### SUMMARY OF THE INVENTION

The present disclosure relates to a system and method for detecting unknown materials such as drugs. More specifically, the present disclosure provides for a system and method for drug detection using SWIR hyperspectral imaging. Most materials of interest show molecular absorption in this region. As used herein, “drugs,” and “drug materials” may refer to illicit and/or non-illicit drugs. The system and method of the present disclosure may hold potential for detecting drug materials on surfaces and in containers and may be applied to detect drug materials in bulk and residue (trace) amounts.

The present disclosure provides for a system and method for the standoff detection of drug materials using infrared, including SWIR, spectroscopic methods. A system may comprise a first collection lens configured to collect a first plurality of interacted photons from a first location comprising an unknown material and a visible imaging device configured to detect the first plurality of interacted photons and generate a visible image. The system may further comprise a second collection lens for focusing and locating a second location comprising at least one unknown material. A second plurality of interacted photons may be collected from the second location. A tunable filter may be configured to filter the second plurality of interacted photons into a plurality of wavelength bands and a SWIR imaging device may detect these photons and generate at least one SWIR hyperspectral image representative of the second location. A processor may be configured to analyze the SWIR hyperspectral image and associate the unknown material with a known material (such as a known drug material).

A method may comprise surveying a first location to identify a second location comprising the unknown material. A plurality of interacted photons from the second location may be collected and filtered into a plurality of wavelength bands. The plurality of interacted photons may be detected and at least one SWIR hyperspectral image may be generated representative of the second location. This SWIR hyperspectral image may be analyzed to associate the unknown material with a known drug material.

In another embodiment, the present disclosure also provides for a non-transitory data storage medium containing program code, which, when executed by a processor, causes the processor to: collect a plurality of interacted photons generated by a second location, filter the interacted photons into a plurality of wavelength bands, detect the plurality of interacted photons and generate at least one SWIR hyperspectral image representative of the second location, and analyze the SWIR hyperspectral image to associate the unknown material with a known drug material.

The system and method provided herein may operate using both passive and active illumination modalities enabling both daytime and nighttime configurations. In addition, the present disclosure contemplates embodiments for the standoff detection of drug materials while operating in either stationary or OTM configurations.

The system and method described herein may also hold potential for enabling automated/aided anomaly detection and enable operators to assess a route/scene of interest, and detect and locate drug materials. The present disclosure also contemplates that a variety of different drug materials may be detected in a scene either simultaneously or sequentially.

### BRIEF DESCRIPTION OF THE DRAWINGS

The accompanying drawings, which are included to provide further understanding of the disclosure and are incorporated in and constitute a part of this specification, illustrate embodiments of the disclosure and, together with the description, serve to explain the principles of the disclosure.

In the drawings:

FIG. 1 is illustrative of a method of the present disclosure.

FIG. 2 is a schematic representation of a system of the present disclosure.

FIG. 3 is a schematic representation of a system of the present disclosure.

FIGS. 4A-4D are illustrative of exemplary packaging options of the systems of the present discourse.

FIGS. 5A-5F are illustrative of the sensitivity capabilities of the system and method of the present disclosure. FIG. 5A is an optical image. FIG. 5B is a NIR chemical image. FIG. 5C is a Raman image and FIG. 5D illustrates bicubic expansion. FIG. 5E is an absorption spectrum and FIG. 5F is a Raman spectrum.

FIGS. 6A-6E are illustrative of the sensitivity capabilities of the system and method of the present disclosure. FIG. 6A is a digital photograph, FIG. 6B is a NIR image, and FIG. 6C is a NIR image. FIG. 6D is an absorption spectrum and FIG. 6E is an absorption spectrum.

FIG. 7 is illustrative of the sensitivity capabilities of the system and method of the present disclosure.

FIG. 8 is illustrative of the sensitivity capabilities of the system and method of the present disclosure.

FIG. 9 is illustrative of the sensitivity capabilities of the system and method of the present disclosure.

FIG. 10A is illustrative of detection of explosive residue on a shoe.

FIG. 10B is illustrative of detection of explosive residue on a car trunk surface.

FIG. 11 is illustrative of the capability of the present disclosure to distinguish between aged and new concrete.

FIG. 12 is illustrative of the capability of the present disclosure to detect disturbed earth.

FIG. 13A is illustrative of a method of the present disclosure that may enable on-the-move detection.

FIG. 13B is illustrative of exemplary integration times of an on-the-move detection configuration of the present disclosure.

FIG. 14 is of on the move detection using a system of the present disclosure.

FIG. 15 is illustrative of the capability of the present disclosure to perform on-the-move detection.

FIG. 16 is illustrative of the capability of the present disclosure to detect and distinguish between multiple materials in a scene.

FIG. 17 is illustrative of the detection capabilities of SWIR technology.

FIG. 18 is illustrative of the detection capabilities of SWIR technology.

FIG. 19 is illustrative of the detection capabilities of SWIR technology.

FIG. 20 is illustrative of the detection capabilities of SWIR technology.

FIG. 21 is illustrative of the detection capabilities of SWIR technology.

FIG. 22 is illustrative of the detection capabilities of SWIR technology.

FIG. 23 is illustrative of the detection capabilities of SWIR technology.

FIG. 24 is illustrative of the detection capabilities of SWIR technology.

FIG. 25 is illustrative of the detection capabilities of SWIR technology.

FIG. 26 is illustrative of the detection capabilities of SWIR technology.

FIG. 27 is illustrative of the detection capabilities of SWIR technology.

FIG. 27 is illustrative of the detection capabilities of SWIR technology.

FIG. 29 is illustrative of the detection capabilities of SWIR technology.

FIG. 30 is illustrative of the detection capabilities of SWIR technology and PLSDA analysis.

FIG. 31 is illustrative of the detection capabilities of SWIR technology and Mahalanobis Distance (MD) analysis.

### DETAILED DESCRIPTION

Reference will now be made in detail to the preferred embodiments of the present disclosure, examples of which are illustrated in the accompanying drawings. Wherever possible, the same reference numbers will be used throughout the drawings to refer to the same or like parts.

The present disclosure provides for a system and method for detecting drug materials using SWIR hyperspectral imaging. The systems and methods of the present disclosure may incorporate or comprise SWIR CONDOR™ and CONDOR-ST technology available from Chemlmage Corporation, Pittsburgh, Pa. and any developments and improvements thereto relating to standoff SWIR technology.

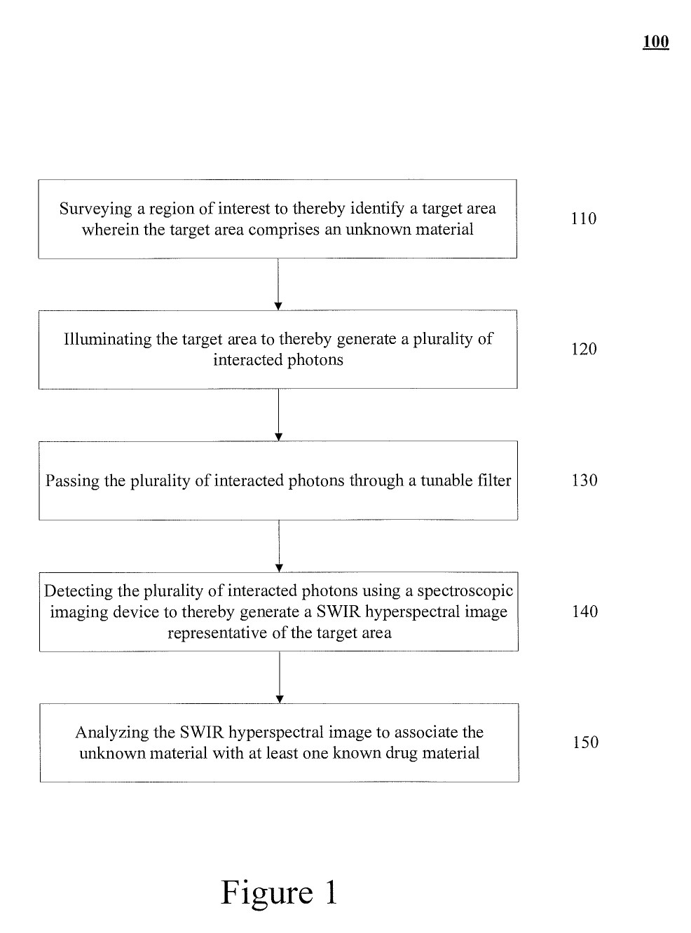

FIG. 1 is representative of a method of the present disclosure. The method 100 may comprise surveying a first location (which may be referred to herein as a region of interest) to thereby identify a second location (which may be referred to herein as a target area) wherein the second location comprises at least one unknown material in step 110. In one embodiment, the first location may be surveyed using a visible imaging device. This visible image device may output a dynamic image of a region of interest in real time and may comprise a video capture device. In another embodiment, the visible imaging device may comprise a RGB camera.

In one embodiment, the second location may be identified based on morphological features. These features may include but are not limited to: size, shape, and color of the second location or of at least one object in the second location.

The present disclosure also contemplates the first location may be surveyed using a SWIR spectroscopic imaging device. In such an embodiment, SWIR hyperspectral imaging may be used to both survey the first location (region of interest) and to locate a second location (a target area) within that first location. The SWIR spectroscopic imaging device may also be used to interrogate the second location to detect and/or identify the unknown material as a drug material.

In step 120 the second location is illuminated to thereby generate a plurality of interacted photons. In one embodiment, the plurality of interacted photons may comprise at least one of: photons reflected by the second location, photons absorbed by the second location, photons scattered by the second location, and photons emitted by the second location. In one embodiment, the interacted photons may be generated by using at least one of: active illumination and passive illumination.

In step 130 the plurality of interacted photons are passed through a tunable filter to filter the interacted photons into a plurality of wavelength bands. The plurality of interacted photons may be detected using a spectroscopic imaging device to thereby generate a SWIR hyperspectral image in step 140. In one embodiment, the SWIR hyperspectral image may comprise a digital image and a spatially resolved SWIR spectrum for each pixel in said image. In one embodiment, the SWIR hyperspectral image may comprise a dynamic chemical image.

The method may further comprise analyzing the SWIR hyperspectral image to thereby associate the unknown material with at least one known drug material in step 150. The unknown material may comprise at least one drug material. When used herein, “drug” or “drug material” may refer to at least one of: an illicit drug material and a non-illicit drug material. Other embodiments may be envisioned that detect other materials of interest including chemicals, biological materials, hazardous materials, and explosives.

In one embodiment, analyzing a SWIR hyperspectral image may comprise comparing at least one of a SWIR hyperspectral image and/or one or more SWIR spectra associated with said SWIR hyperspectral image with a reference data base wherein the reference data base comprises at least one reference SWIR data set associated with a known material, such as a known drug material. In one embodiment, the reference data base may also comprise at least one reference visible data set associated with a known material or object. This reference data base may be consulted during surveying of a first location.

Comparing the SWIR hyperspectral image (or a visible image) to a reference data set may be accomplished using one or more algorithmic techniques. These techniques may comprise at least one chemometric and/or ratiometric techniques (such as wavelength division). Chemometric techniques may include, but are not limited to: principle components analysis (PCA), PLSDA, cosine correlation analysis, Euclidian distance analysis, k-means clustering, multivariate curve resolution, band t. entropy method, MD, adaptive subspace detector, spectral mixture resolution, Bayesian fusion, and combinations thereof.

In one embodiment, the method may further provide for data fusion in which data generated by two or more different spectroscopic imaging modalities may be fused. This fusion may be accomplished by applying at least one fusion algorithm known in the art. The present disclosure contemplates a variety of different fusion combinations including at least two of the following: a visible image, a SWIR hyperspectral image, a MWIR hyperspectral image and a LWIR hyperspectral image may be generated.

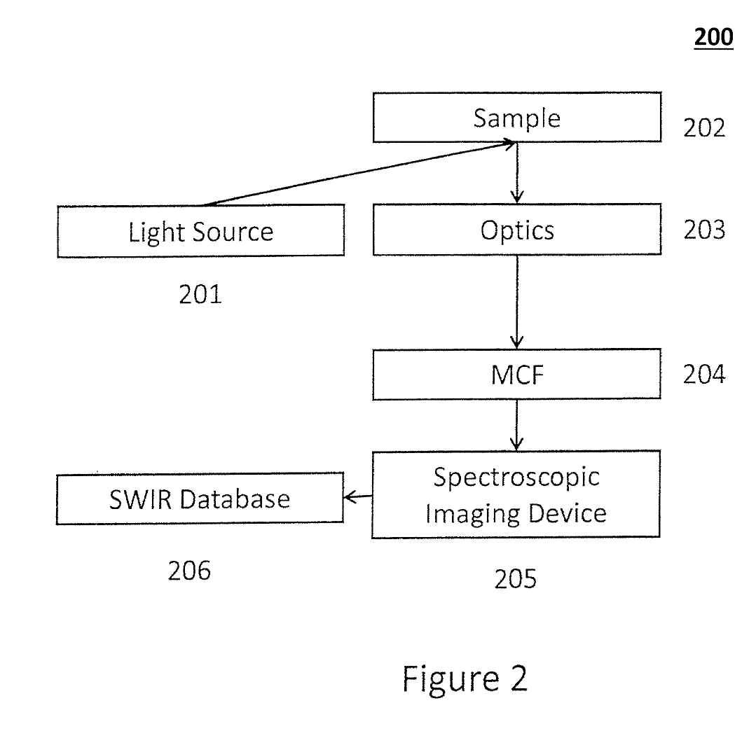

The present disclosure also provides for a system for detecting and/or identifying drugs and/or other materials. FIG. 2 is a schematic representation of a system of the present disclosure. The system 200 may comprise an illumination light source 201 configured to illuminate an unknown sample 202 to thereby generate a plurality of interacted photons. In one embodiment, the illumination light source 201 may comprise at least one of: a laser illumination source, a broadband light source, and an ambient light source. In one embodiment, the system 200 may be configured for passive illumination and/or active illumination.

In one embodiment, at least one illumination source will incorporate IR long pass filters to eliminate any visible light emitted from the source(s) and allow for only IR light to illuminate the scene. The IR light is eye safe and invisible to visible sensors. For daytime operation, one embodiment provides for the use of the sun as an illumination source. In an embodiment for nighttime operation using active illumination, a set of tungsten white light illumination sources may be used. Tungsten white light alone is eye safe but is not invisible to visible sensors. By coupling the tungsten white light sources with IR long pass filters all visible light will be blocked and only IR light will illuminate the scene. In one embodiment, four (4) spotlights with 5900 lumens each, with 6° angular divergence may produce an average intensity of about 1100 and about 5 m illumination diameter at a 50 m standoff distance. Additional lighting may be used to carry out measurements at standoff distances of 200-1000 m.

Interacted photons generated by illuminating the second location may be collected by one or more optics 203. In one embodiment, telescope optics may be configured for at least one of: locating and focusing on a second location and/or collecting a plurality of interacted photons. In one embodiment, a telescope optics may be implemented to enable magnification and thereby SWIR hyperspectral imaging sensitivity.

The interacted photons may be passed through a tunable filter 204. The tunable filter in FIG. 2 is illustrated as a multi-conjugate liquid crystal tunable filter (MCF) 204. In one embodiment, MCF technology available from ChemImage Corporation, Pittsburgh, Pa. may be used. A MCF, a type of LCTF, consists of a series of stages composed of polarizers, retarders and liquid crystals. The MCF is capable of providing diffraction limited spatial resolution, and a spectral resolution consistent with a single stage dispersive monochromator. A MCF may be computer controlled with no moving parts. It may be tuned to any wavelength in the given filter range. This results in an essentially infinite number of spectral bands available. A MCF provides high optical throughput, superior out-of-band rejection and faster tuning speeds. While images associated with spectral bands of interest must be collected individually, material-specific chemical images revealing target detections may be acquired, processed and displayed in numerous times each second. Combining MCF technology with software targeting algorithms holds great potential for detecting drug materials using SWIR hyperspectral imaging, including potential for OTM detection.

This technology is more fully described in the following U.S. patents and patent applications: U.S. Pat. No. 6,992,809, filed on Jan. 31, 2006, entitled “Multi-Conjugate Liquid Crystal Tunable Filter,” U.S. Pat. No. 7,362,489, filed on Apr. 22, 2008, entitled “Multi-Conjugate Liquid Crystal Tunable Filter,” Ser. No. 13/066,428, filed on Apr. 14, 2011, entitled “Short wave infrared multi-conjugate liquid crystal tunable filter.” These patents and patent applications are hereby incorporated by reference in their entireties.

The MCF may be used to filter light to the spectroscopic imaging device 205 and is capable of tuning to an infinite number of spectral bands. Therefore, for nighttime operation using active broadband IR illumination, decreasing spectral resolution may not be necessary. Nighttime operation of the system may cover the same spectral range and is capable of the same number of spectral bands as daytime operation. Transition from daytime to nighttime operations should be as simple as switching on a lamp.

The present disclosure is not limited to the use of a MCF and contemplates that the tunable filter 204 may comprise at least one of: a SWIR multi-conjugate liquid crystal tunable filter, a SWIR liquid crystal tunable filter, a Fabry Perot angle tuned filter, an acousto-optic tunable filter, a liquid crystal tunable filter, a Lyot filter, an Evans split element liquid crystal tunable filter, a Solc liquid crystal tunable filter, a fixed wavelength Fabry Perot tunable filter, an air-tuned Fabry Perot tunable filter, a mechanically-tuned Fabry Perot tunable filter, and a liquid crystal Fabry Perot tunable filter.

The plurality of interacted photons may detected using a spectroscopic imaging device 205. The spectroscopic imaging device may be configured to generate a SWIR hyperspectral image representative of the second location interrogated (which comprises the unknown material). In another embodiment, the spectroscopic imaging device 205 may be configured so as to generate at least one of: a plurality of spatially resolved SWIR images, a plurality of spatially resolved SWIR spectra, a SWIR chemical image, and combinations thereof.

The system 200 may further comprise a reference database 206 comprising at least one SWIR reference data set. A processor may be configured to access this SWIR database 206 to analyze a SWIR hyperspectral image.

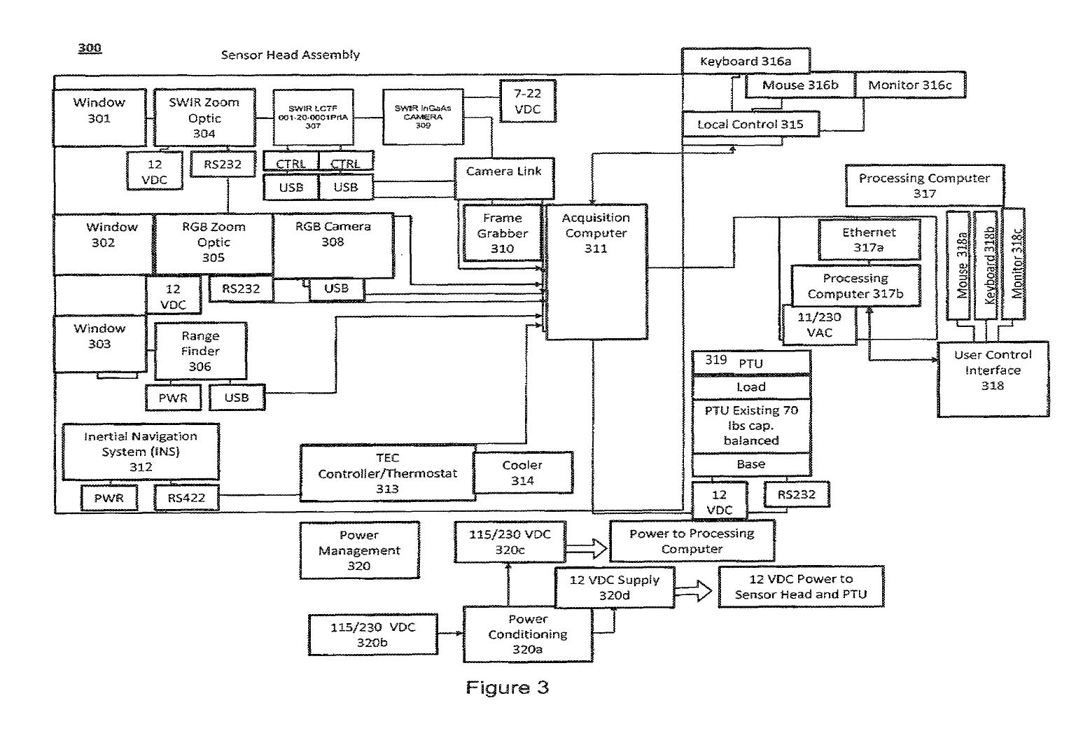

FIG. 3 is a more detailed schematic of a system of the present disclosure. The system 300 may comprise one or more windows 301, 302, and 303, which may also be referred to as collection lenses, or lenses, herein. The system may comprise a one or more zoom optics 304, 305. In one embodiment, a SWIR zoom optic 304 may be operatively coupled to a tunable filter 307. In FIG. 3, the tunable filter is illustrated as a SWIR LCTF 307. However, the tunable filter 307 may comprise any filter contemplated herein. The SWIR LCTF 307 may be configured to effectively separate a plurality of interacted photons into a plurality of wavelength bands. The plurality of interacted photons may be detected by a SWIR detector, illustrated in FIG. 3 as a SWIR InGaAs Camera 309. However, other embodiments may comprise other detectors known in the art including but not limited to a mercury cadmium telluride (MCT) detector, a CCD detector, an intensified charged coupled device (ICCD), a indium antimonide (InSb) detector, and an InGaAs detector. In one embodiment a SWIR detector 309 may be operatively coupled to a frame grabber 310 which may operate to capture image frames generated by the detector 309.

The system 300 may further comprise a visible zoom optic, illustrated in FIG. 3 as a

RGB zoom optic 305. This RGB zoom optic 305 may be operatively coupled to visible detector, illustrated as an RGB camera 308. However, this visible detector may also comprise a video capture device.

The system 300 may further comprise a number of controls and additional features to enable navigation, selection of a location, and overall operation and management of the system 300. The system 300 may comprise a range finder 306 which may be configured to measure distance to a specific location or object. In one embodiment, at least one of a frame grabber 310, a RGB camera 308, a range finder 306, and an inertial navigation system 312 may be operatively coupled to an acquisition computer 311. This acquisition computer 311 may be coupled to at least one of: a local control 315, a processing computer 317, and a PTU 319. In one embodiment, a local control 315 may comprise a computer and further comprise at least one of: a keyboard 316a, a mouse 316b, and a monitor 316c. In one embodiment, a processing computer 317 may comprise at least one of: an Ethernet configuration 317a, and a second processing computer 317b. The processing computer 317 may be operatively coupled to a user control interface 318. The user control interface 318 may comprise at least one of: a mouse 318a, keyboard 318b, and monitor 318c. The system may further comprise a power management system 320 which may be operatively coupled to the system 300.

In one embodiment, the system of the present disclosure may incorporate a high pixel resolution, high frame rate color video camera system to assist in locating targets of interest. This may be represented in FIG. 3 as a RGB camera 308. The SWIR HSI portion of the system may consist of an InGaAs focal plane camera coupled to a wavelength-agile MCF in combination with a zoom optic capable of viewing a large area, or imaging a localized area at high magnification. In one embodiment of operation, an area would first be screened using the wide field setting on the zoom lens. Once the area is screened and potential targets are identified, confirmation of the area may be accomplished as necessary by using the narrow field setting on the zoom lens.

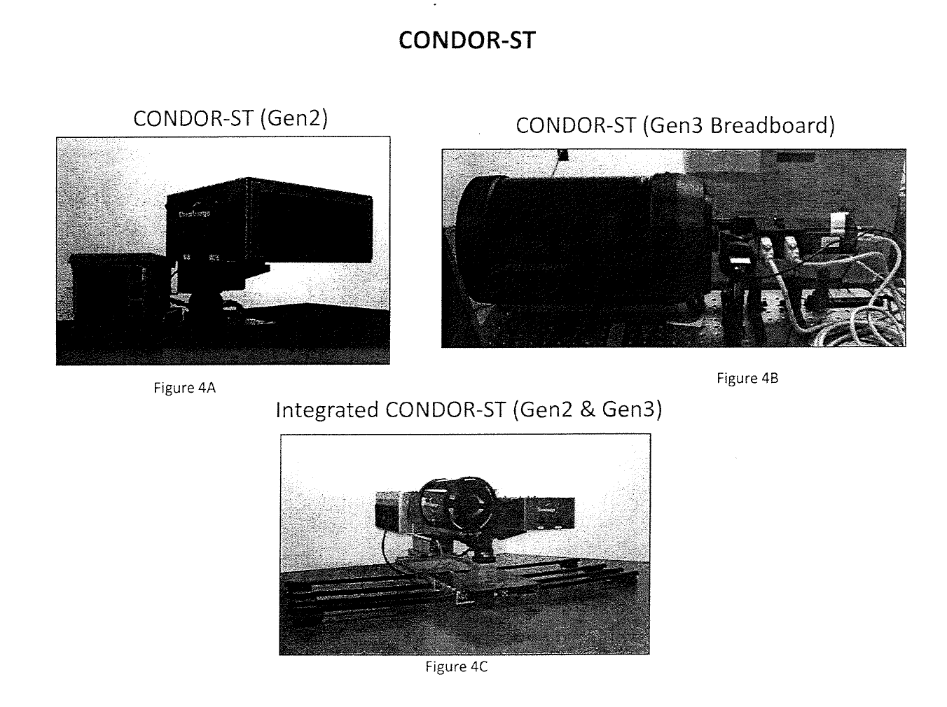

FIGS. 4A-4D are illustrative of exemplary embodiments of packaging of the systems of the present disclosure. In one embodiment, a 20x magnification increase may be used to increase SWIR HSI detection sensitivity. In one example, sensitivity may be increased by integration of an 8″ diameter telescope.

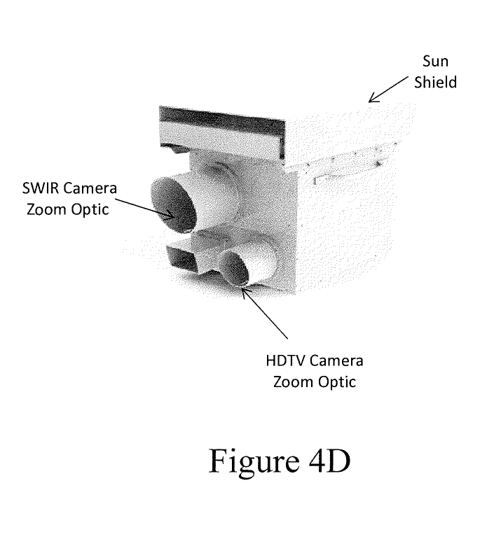

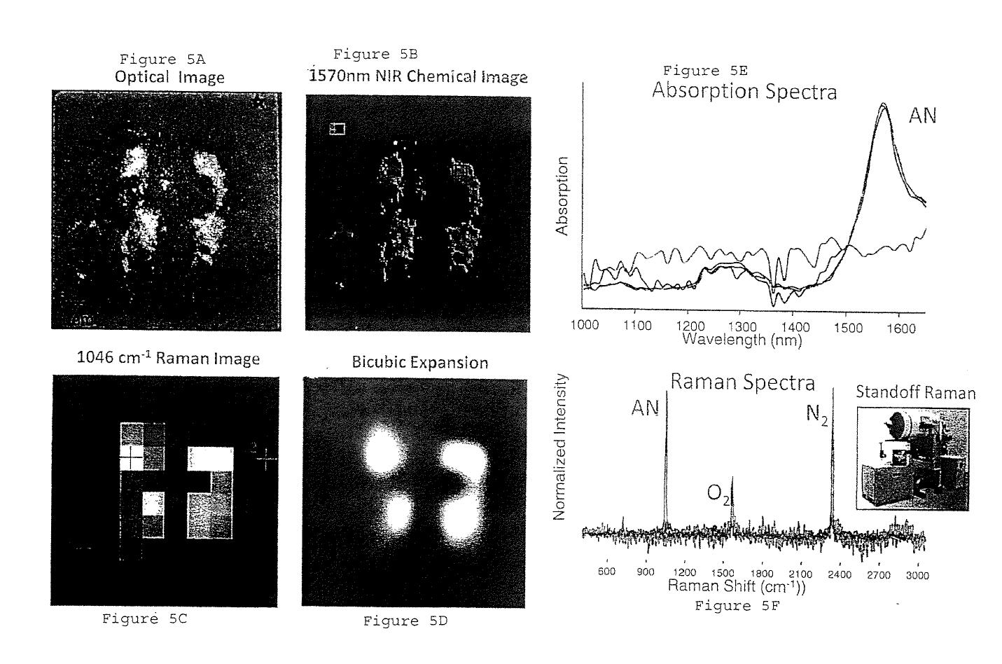

FIGS. 5A-5E illustrate the detection capabilities of the present disclosure for unknown materials. While FIGS. 5A-5E illustrate the detection of ammonium nitrate (AN), they are provided to show how the system and method of the present disclosure may be applied to detecting and identification of any unknown material. Therefore, a similar analysis may be used to detect drug materials. FIG. 5A depicts an exemplary optical image, FIG. 5B depicts a NIR chemical image, a Raman image is illustrated in FIG. 5C, and Bicubic Expansion is illustrated in FIG. 5D. While the present disclosure focuses on the use of SWIR hyperspectral image and spectroscopy, these figures illustrate the potential of also applying other techniques. Absorption spectra and Raman spectra are depicted in FIGS. 5E and 5F, respectively. FIGS. 6A-6E further illustrate the potential of a system and method of the present disclosure for detecting unknown materials. These Figures illustrate AN detection, but a similar analysis may be applied to detecting drug materials. FIG. 6A depicts an exemplary digital photograph, FIG. 6B illustrates a NIR Image and a NIR image is also presented in FIG. 6C. Absorption spectra are depicted in FIGS. 6D and 6E, respectively.

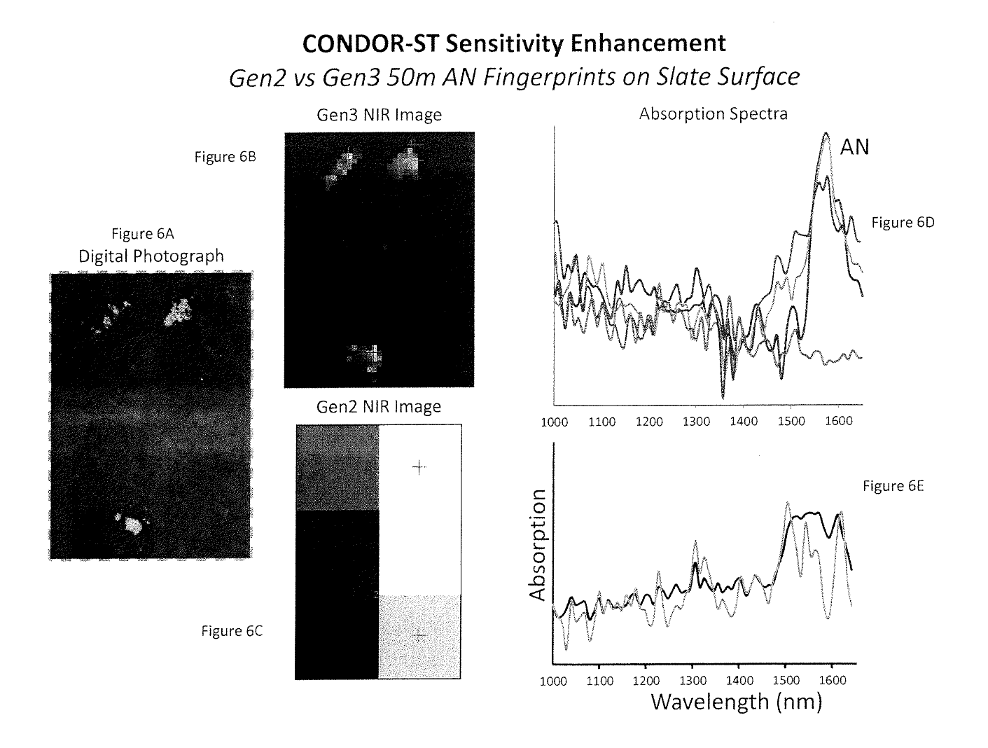

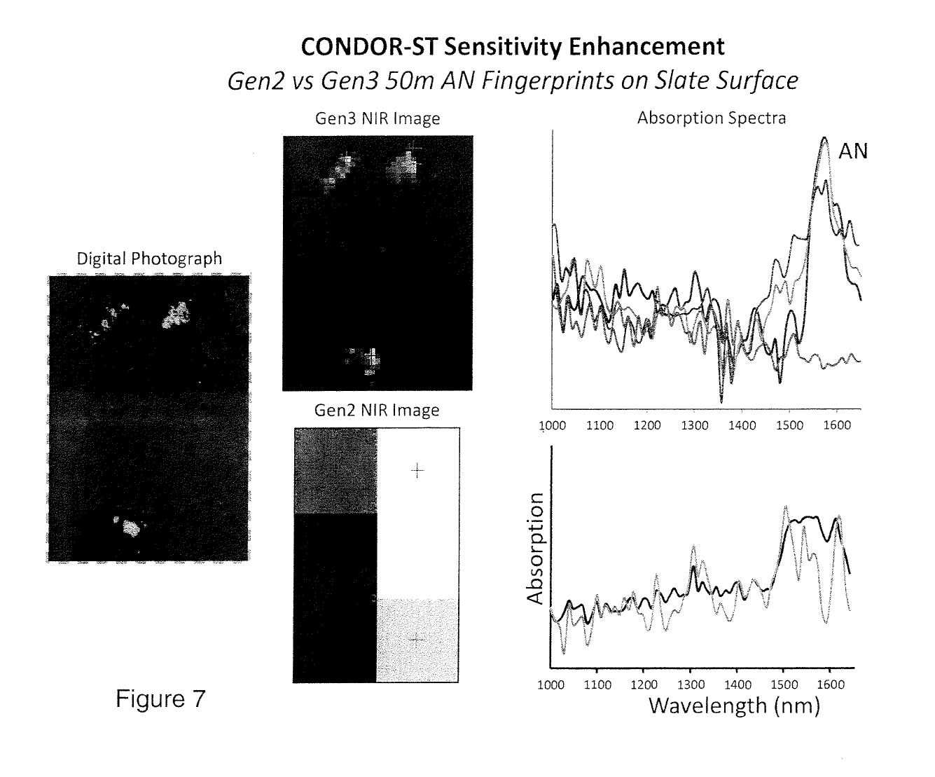

FIGS. 7-12 provide further support of the detection capabilities of the present disclosure. Included in these Figures is evidence of the sensitivity enhancement capabilities of the system and method of the present disclosure. This data demonstrates AN, but the analysis can also be applied to embodiments detecting drug materials on various surfaces. FIG. 7 is illustrative of the detection of AN on fingerprints on a slate surface obtained using a Gen3 sensor at 50 m standoff distance. FIG. 8 is illustrative of a comparison between Gen2 and Gen3 sensors. The comparison is illustrative of the detection of AN on fingerprints on a slate surface at 50 m standoff distance.

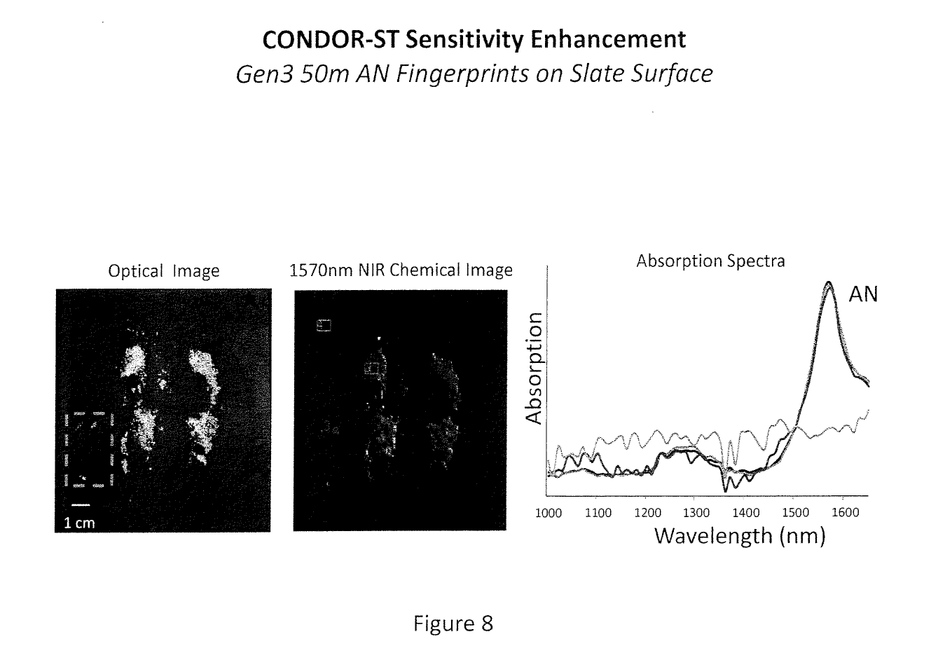

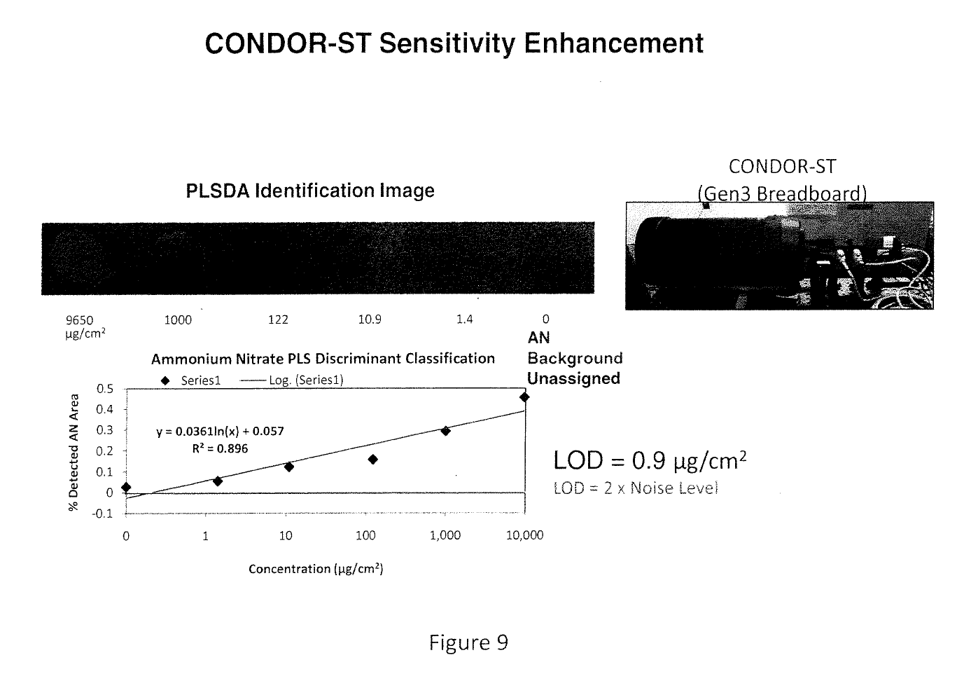

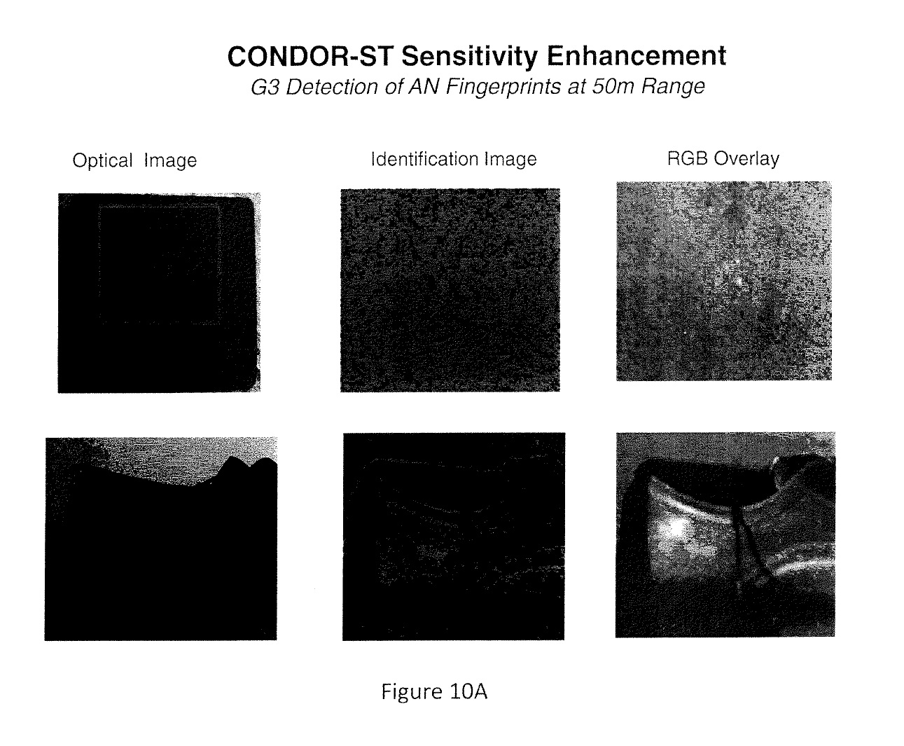

FIG. 9 is also illustrative of CONDOR-ST sensitivity enhancements. By increasing magnification of the image gathering optics, sensitivity of the CONDOR-ST SWIR HSI system can be increased. The sample in FIG. 9 comprises AN on substrates (aluminum sheet metal, slate tile, dust/dirt covered slate tile, shoe) by fingerprint transfer. The sensor used to obtain the results was a CONDOR-ST (Gen3) sensor with an 8″ diameter telescope. FIG. 10A is illustrative of the detection of AN fingerprints at 50 m standoff range on a shoe. This illustrates the potential of SWIR hyperspectral imaging for detection of unknown materials on a variety of surfaces. Such application is more fully described in U.S. patent application Ser. No. 12/754,229, filed on Apr. 5, 2010, entitled “Chemical Imaging Explosives (CHIMED) Optical Sensor using SWIR”, which is hereby incorporated by reference in its entirety.

FIG. 10B is illustrative of detection of AN fingerprint residue transferred by touching a car trunk surface. This data was obtained at 20 m standoff range in real-time. A similar scenario may be applied to the detection of drug materials. For example, when a vehicle is suspected of carrying drug materials, various locations of interest on the vehicle may be selected and interrogated using SWIR hyperspectral imaging. These locations of interest may include a door handle or the trunk/storage area of the vehicle.

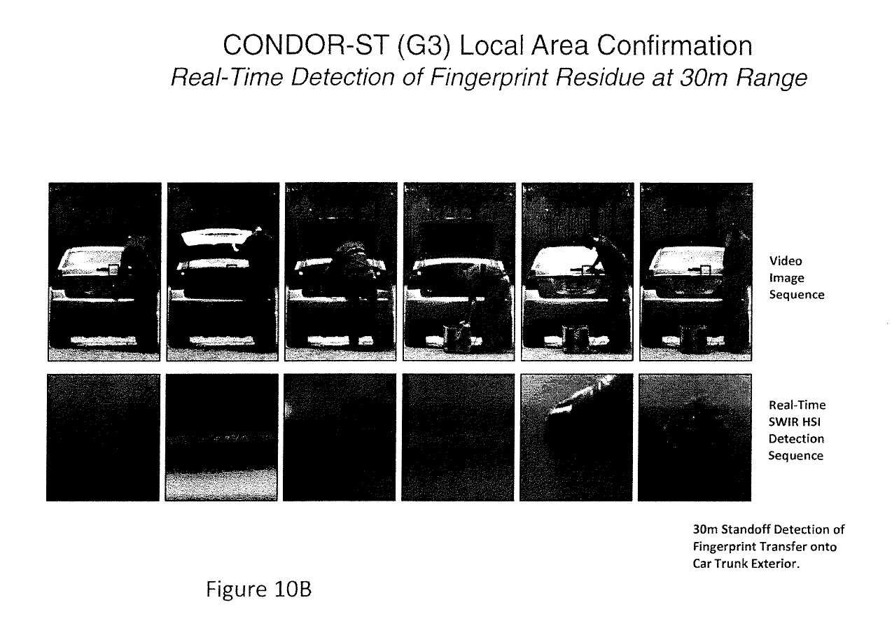

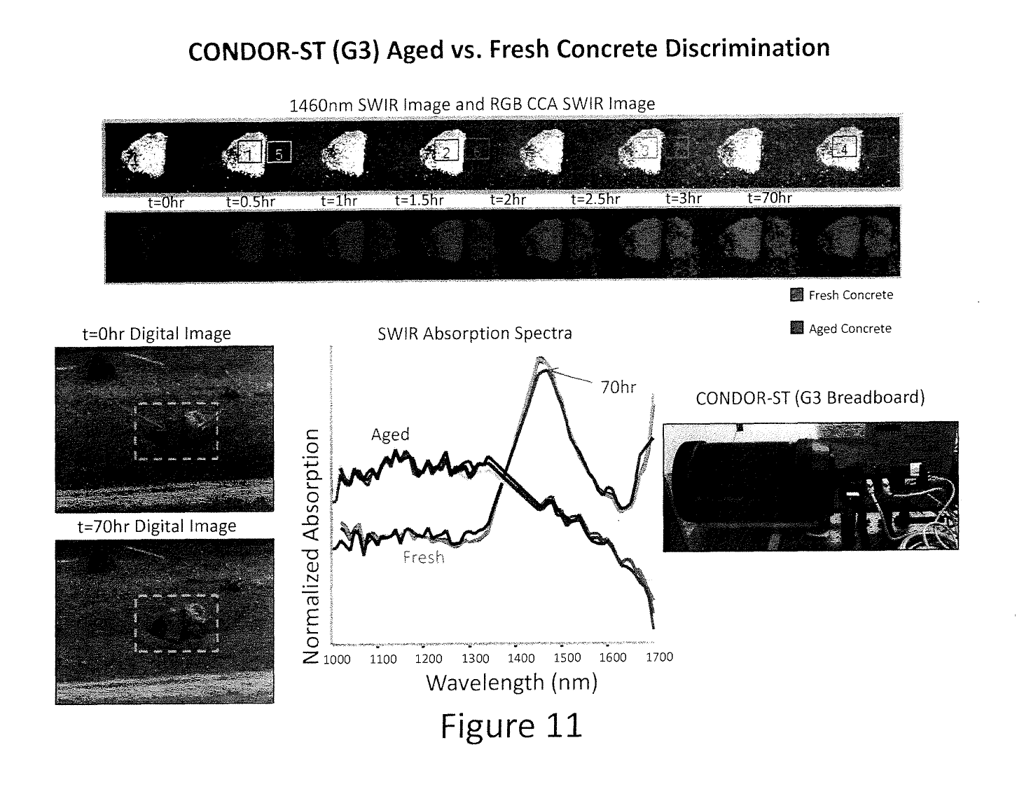

FIG. 11 is illustrative of the ability of the system and method of the present disclosure to detect between aged and fresh concrete. FIG. 12 is illustrative of the ability of the system and method of the present disclosure to detect disturbed earth at a 200 m standoff range. These figures are included to further support the detection capabilities of SWIR hyperspectral imaging for unknown materials on a variety of different surfaces and locations where drug material may be found.

In one embodiment, the systems and methods of the present disclosure may be configured to operate in at least one of the following configurations: proximal detection, standoff detection, stationary detection, and on-the-move detection. Standoff detection of explosives is more fully described in the following U.S. patents and patent applications, which are hereby incorporated by reference in their entireties: U.S. Pat. No. 7,692,775, filed on Jun. 9, 2006, entitled “Time and Space Resolved Standoff Hyperspectral IED Explosives LIDAR Detection”, Ser. No. 12/199,145, filed on Aug. 27, 2008, entitled “Time and Space Resolved Standoff Hyperspectral IED Explosives LIDAR Detection”, Ser. No. 12/802,994, filed on Jun. 17, 2010, entitled “SWIR Targeted Agile Raman (STAR) System for Detection of Emplace Explosives.”

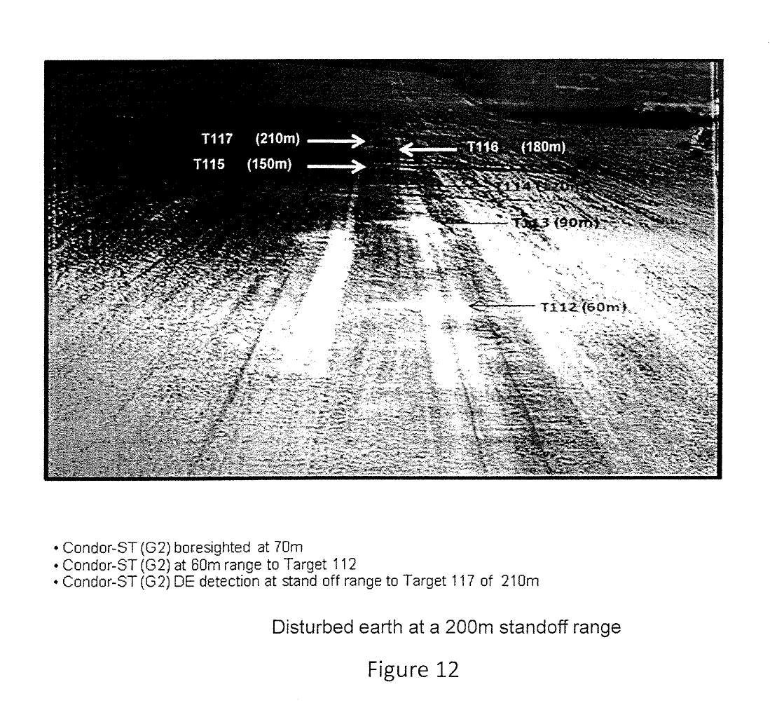

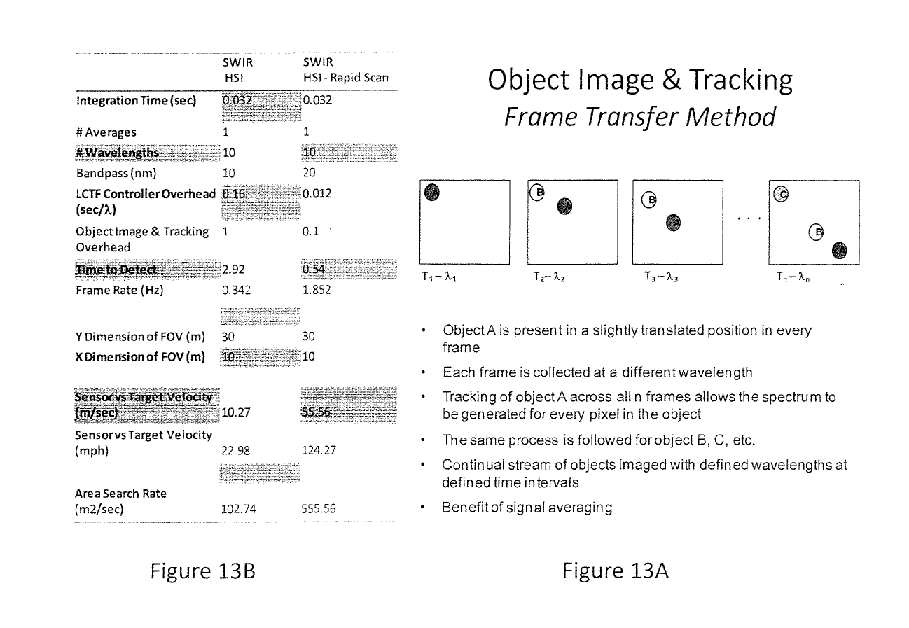

In one embodiment, the system of the present disclosure may be used for stationary and OTM drug detection, explosive detection, disturbed earth detection and camouflage concealment and detection. In one embodiment, OTM detection may be enabled by using dynamic imaging in one or more modalities including visible and SWIR. FIGS. 13A and 13B are provided to further explain OTM detection according to one embodiment of the present disclosure. The present disclosure also provides for a system and method of dynamic chemical imaging in which more than one object of interest passes continuously through the FOV. Such continuous stream of objects, results in the average amount of time required to collect all frames for a given object being equivalent to the amount of time to capture one frame as the total number of frames under collection approaches infinity (frame collection rate reaches steady state). In other words, the system is continually collecting the frames of data for multiple objects simultaneously and with every new frame, the set of frames for any single object is completed. In one embodiment, the objects of interest are of a size substantially smaller than the FOV to allow more than one object to be in the FOV at any given time. Referring to FIG. 13A, OTM detection may be enabled by collecting each frame at a different wavelength. One or more objects may be present in slightly translated positions in each image frame acquired. Tracking of objects across all n frames allows the spectrum to be generated for each pixel in the object. The same process may be followed for all objects in the frames. A continual stream of objects will be imaged with defined wavelengths at defined time intervals. This methodology may also utilize the benefits of signal averaging. FIG. 13B is provided to illustrate approximate integration times associated with the configuration of FIG. 13A.

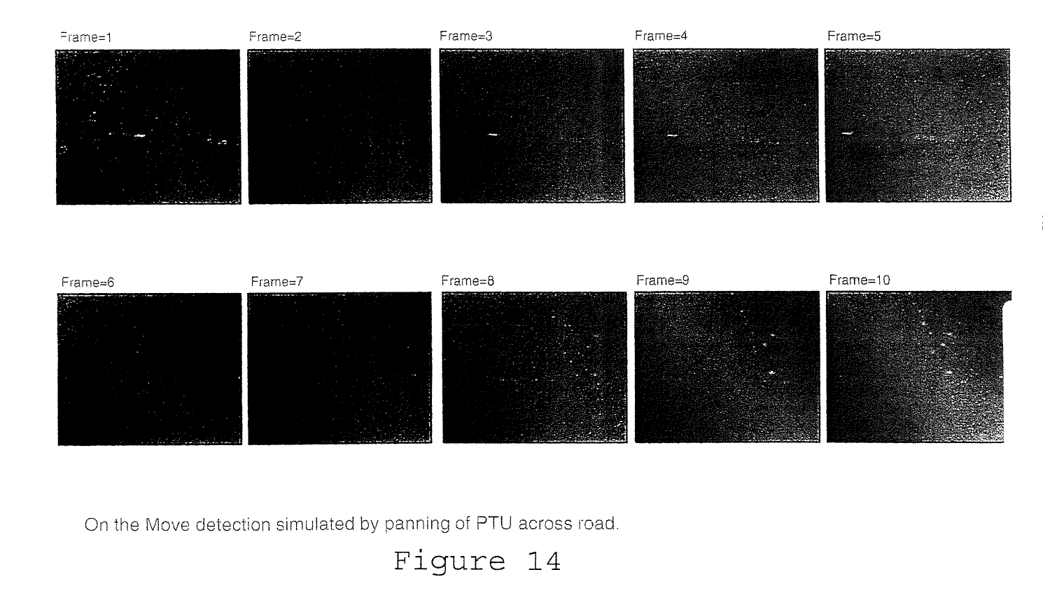

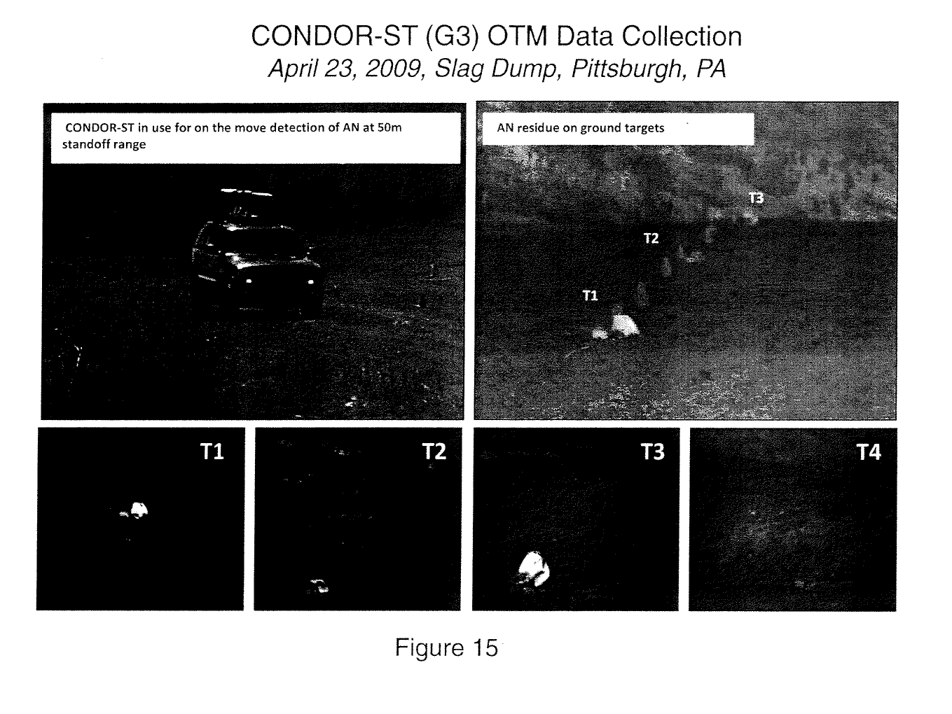

FIG. 14 is illustrative of OTM detection simulated by panning of PTU across a road. FIG. 15 is illustrative of OTM of AN residue deposited on the ground at a standoff range of >50 m. The data was collected while moving at 3-5 mph. A similar approach may be applied to detecting drug materials.

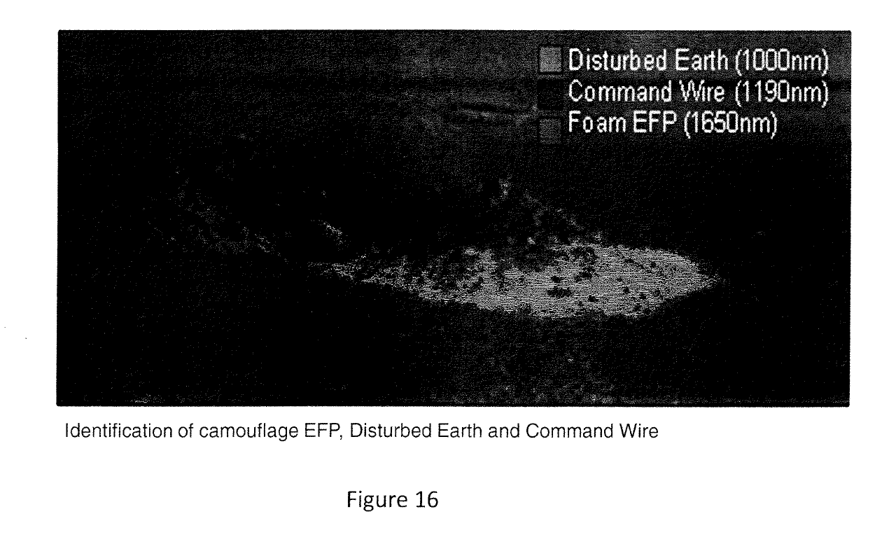

Another example wherein different materials detected in a scene can be assigned different pseudo colors for easy discrimination between materials is illustrated by FIG. 16. Here disturbed earth, command wire, and foam are all detected and assigned different pseudo colors. Drug materials may also be detected and discriminated from other materials in a scene. Pixels containing the materials of interest may be pseudo colored to indicate positive detection. The use of pseudo color enhancement is more fully described in U.S. patent Ser. No. 12/799,779, filed on Apr. 30, 2010, entitled “System and Method for Component Discrimination Enhancement based on Multispectral Addition Imaging,” hereby incorporated by reference in its entirety.

The present disclosure contemplates the system and method disclosed herein may be configured so as to enable integration with LWIR, MM Wave, and/or GPR sensors via industry standard fusion software. In one embodiment, this fusion software may comprise Chemlmage's FIST (“Forensic Integrated Search”) technology, available from Chemlmage Corporation, Pittsburgh, Pa. This technology is more fully described in pending U.S. patent application Ser. Nos. 11/450,138, filed on Jun. 9, 2006, entitled “Forensic Integrated Search Technology”; Ser. No. 12/017,445, filed on Jan. 22, 2008, entitled “Forensic Integrated Search Technology with Instrument Weight Factor Determination”; Ser. No. 12/196,921, filed on Aug. 22, 2008, entitled “Adaptive Method for Outlier Detection and Spectral Library. Augmentation”; and Ser. No. 12/339,805, filed on Dec. 19, 2008, entitled “Detection of Pathogenic Microorganisms Using Fused Sensor Data”. Each of these applications is hereby incorporated by reference in their entireties.

The present disclosure also contemplates the incorporation of real-time anomaly detection and classification algorithms in a software package associated with the sensor. In such an embodiment, the system will have the ability to perform autonomous detection of a wide variety of targets. Such an embodiment provides for a single sensor system to support automated counter mine algorithms, aided target cuing, Aided Target Recognition (AiTR) of difficult targets, and anomaly detection and identification in complex/urban areas.

In another embodiment, the present disclosure provides for ChemFusion Improvements. Such improvements include the use of grid search methodology to establish improved weighting parameters for individual sensor modality classifiers under JFIST Bayesian architecture. Improvements in Pd and Pfa can be realized by full execution of combinatorial decision making applied to multiple detections afforded by hyperspectral imaging. In another embodiment, image weighted Bayesian fusion may be used.

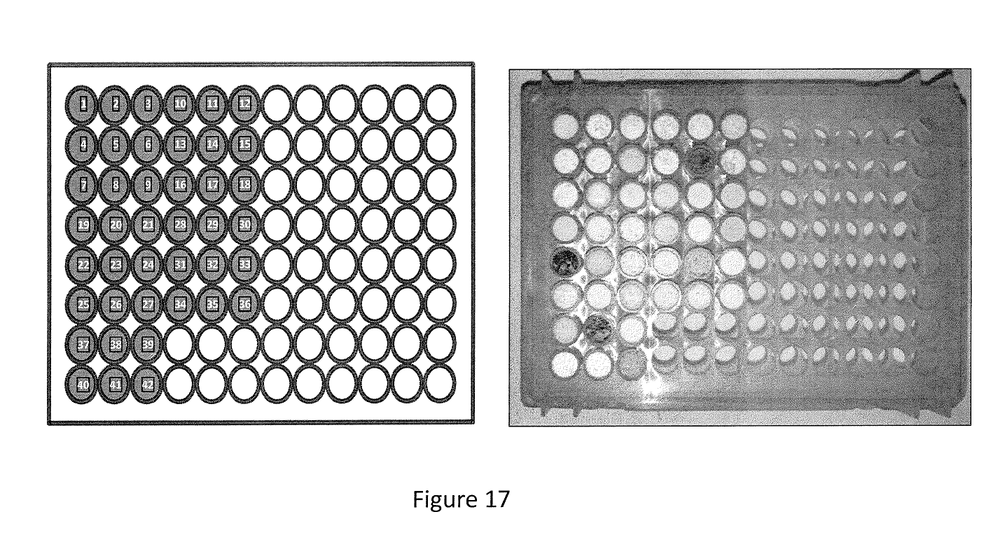

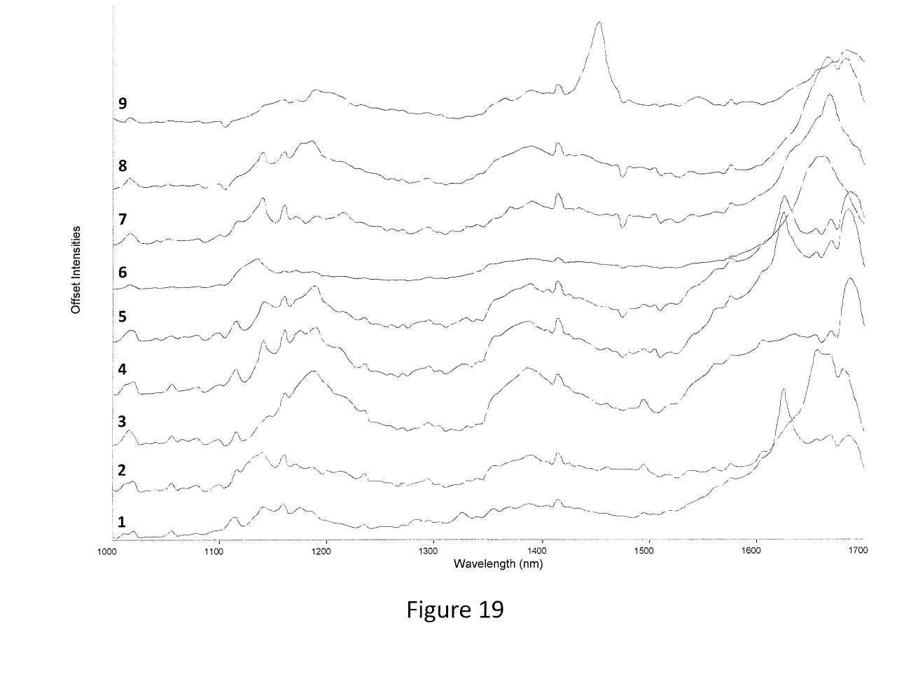

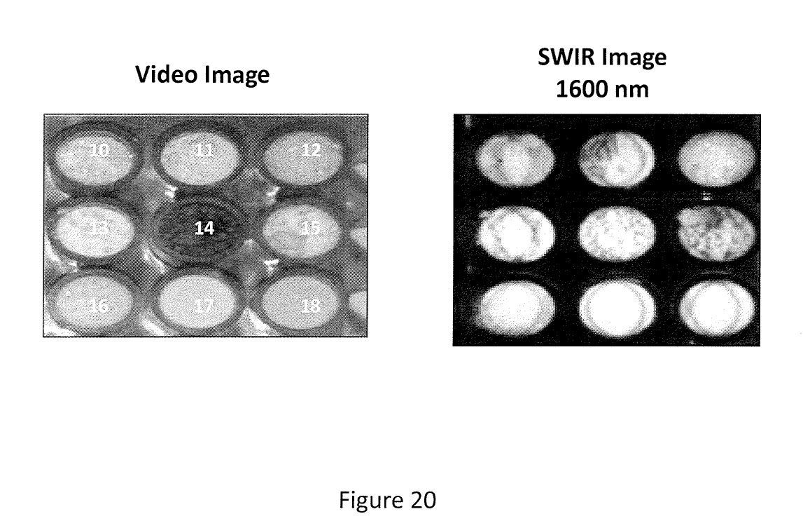

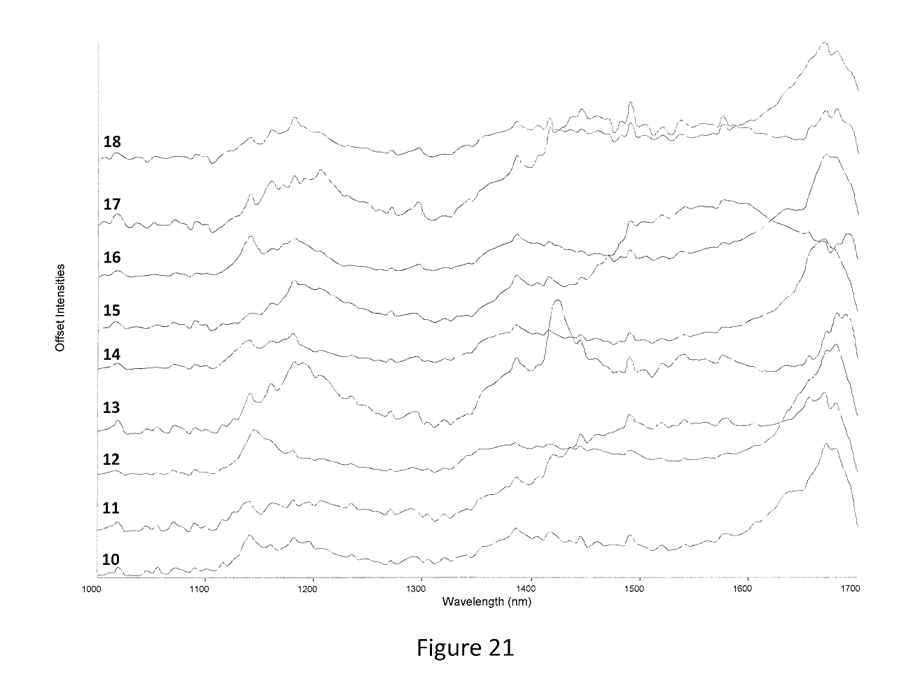

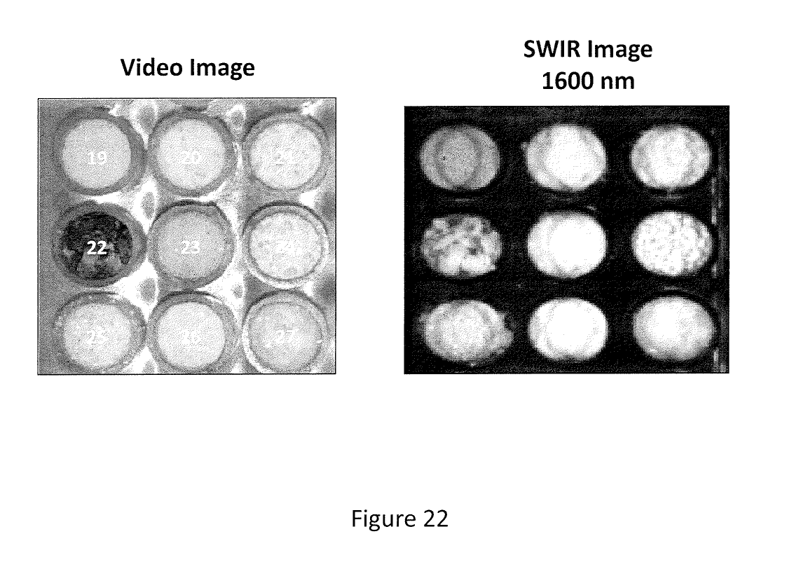

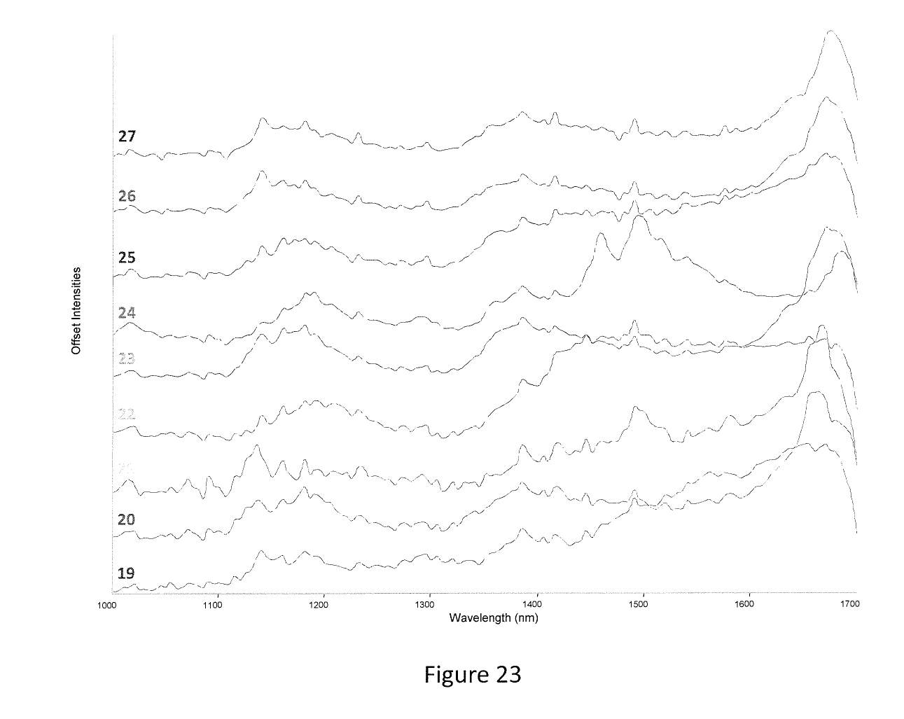

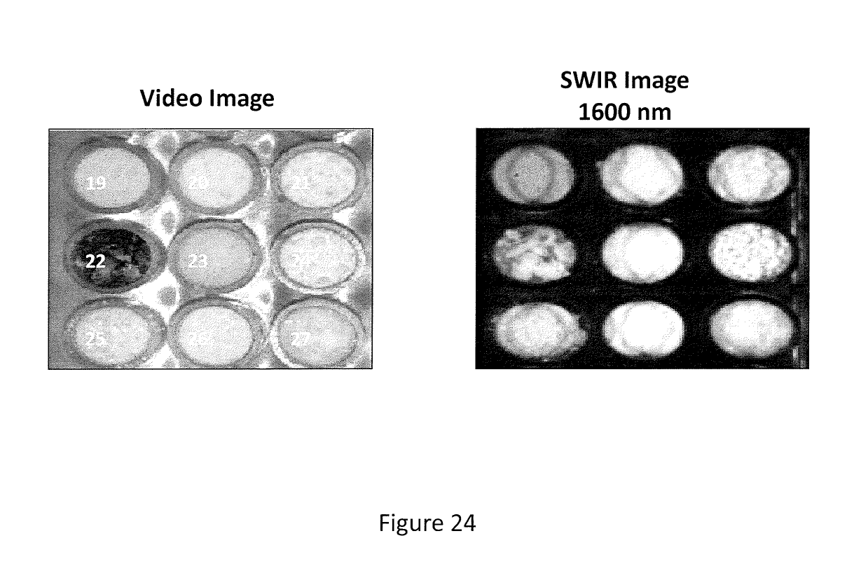

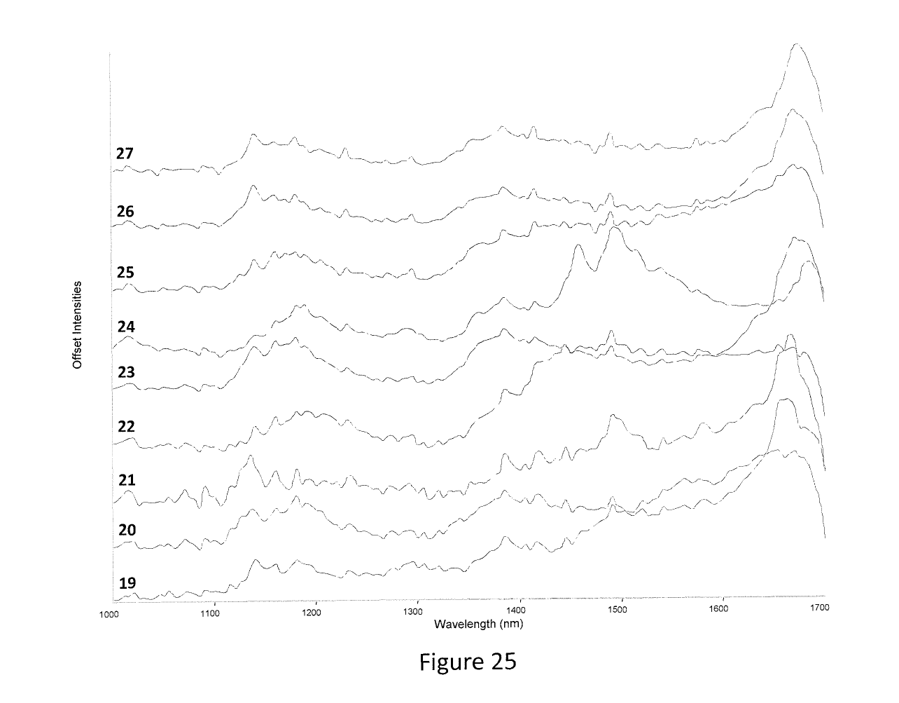

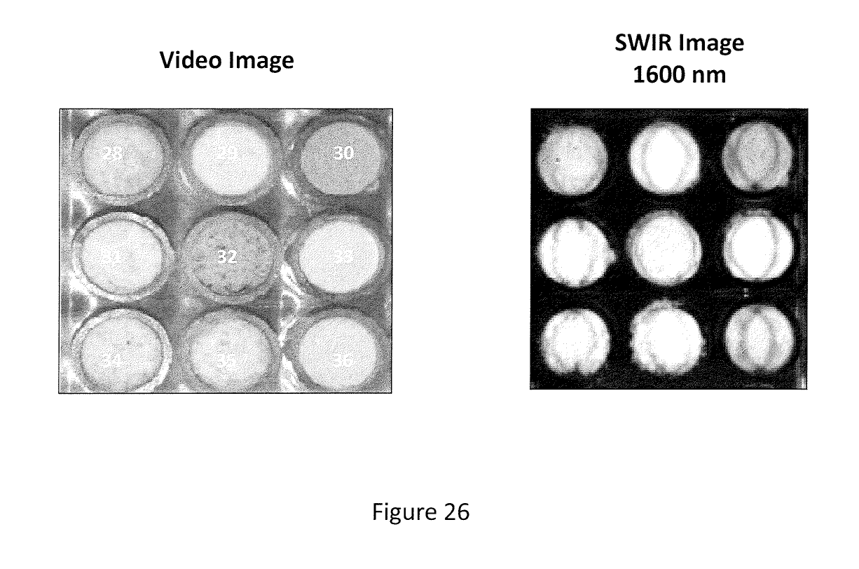

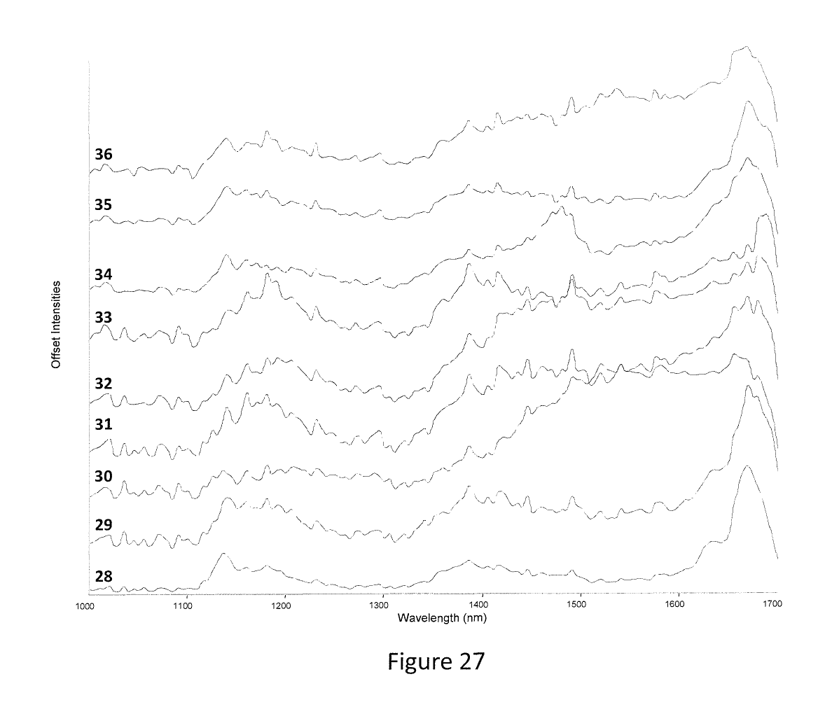

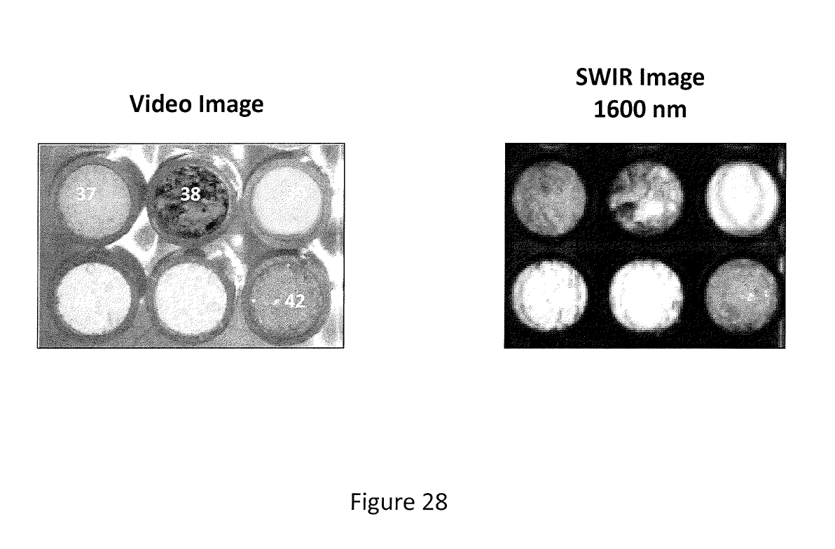

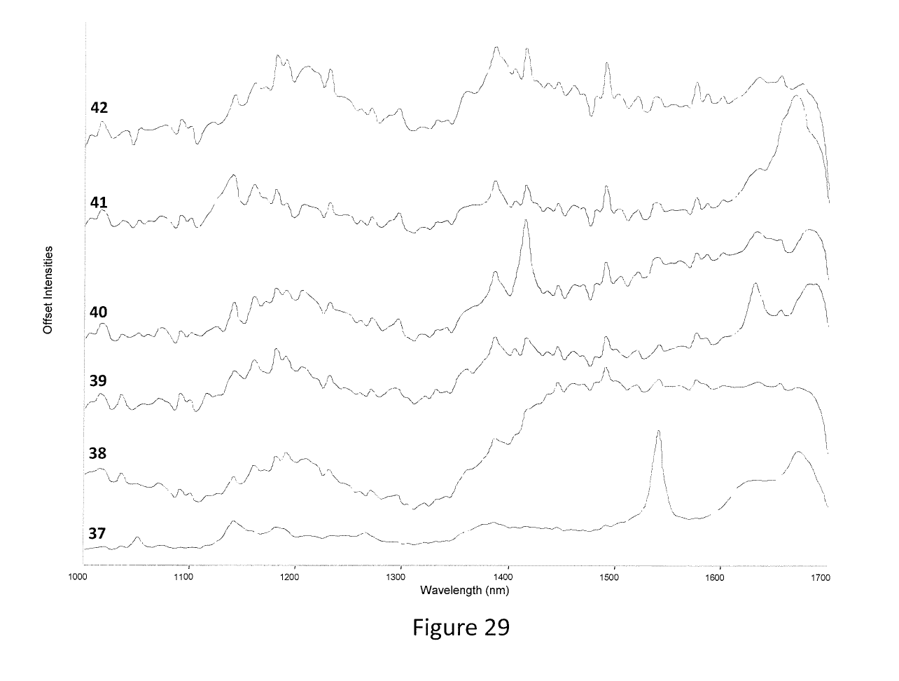

In one embodiment, the system and method of the present disclosure may relate specifically to the use of SWIR technology for drug detection. Examples of the detection capabilities of the present disclosure are provided in FIGS. 17-31 and illustrate the detection capabilities of SWIR technology. This data was generated using SWIR CONDOR™ technology, available from Chemlmage Corporation, Pittsburgh, Pa. and illustrates the ability of SWIR to detect various drug materials.

Various samples were deposited for analysis as shown in FIG. 17. Table 1 below illustrates the various drug samples and their corresponding locations in FIG. 17.

TABLE 1

| Location | Drug Material |
|---|---|
| 1 | Allobarbital |
| 2 | Alprazolam |
| 3 | Amobarbital |
| 4 | Aprobarbital |
| 5 | Butalbital |
| 6 | Chlordiazepoxide |
| 7 | Clonazepam |
| 8 | Cocaine (base) |
| 9 | Codeine |
| 10 | D-amphetamine sulfate |
| 11 | Diazepam |
| 12 | Diphenhydramine |
| 13 | Fluoxymesterone |
| 14 | Flurazepam di-HCl |
| 15 | Gamma-hydroxybutyric acid |
| 16 | Glutethimide |
| 17 | Hexobarbital |
| 18 | Hydromorphone HCl |
| 19 | Hydroxyamphetamine |
| 20 | Ketamine |
| 21 | Lorazepam |
| 22 | Marijuana |
| 23 | Meperidine HCl |
| 24 | Meprobamate |
| 25 | Mescaline |
| 26 | Methadone HCl |
| 27 | Methamphetamine HCl |
| 28 | Methaqualone |
| 29 | Methylphenidate HCl |
| 30 | Oxazepam |
| 31 | Oxycodone HCl |
| 32 | Pentazocine |
| 33 | Pentobarbital |
| 34 | Phendimetrazine bitartrate |
| 35 | Phenmetrazine HCl |
| 36 | Phenobarbital |
| 37 | Pseudoephedrine |
| 38 | Psilocyn |
| 39 | Secobarbital |
| 40 | Stanozolol |
| 41 | Triazolam |
| 42 | Blank (Silica Only) |

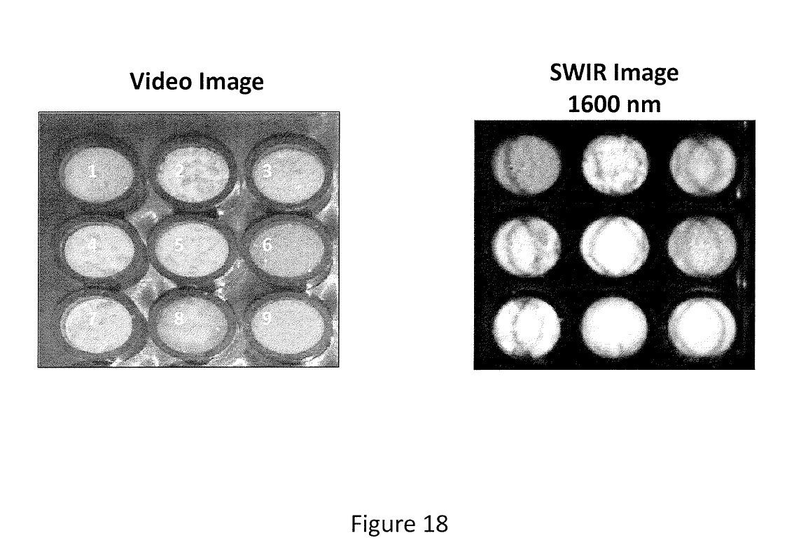

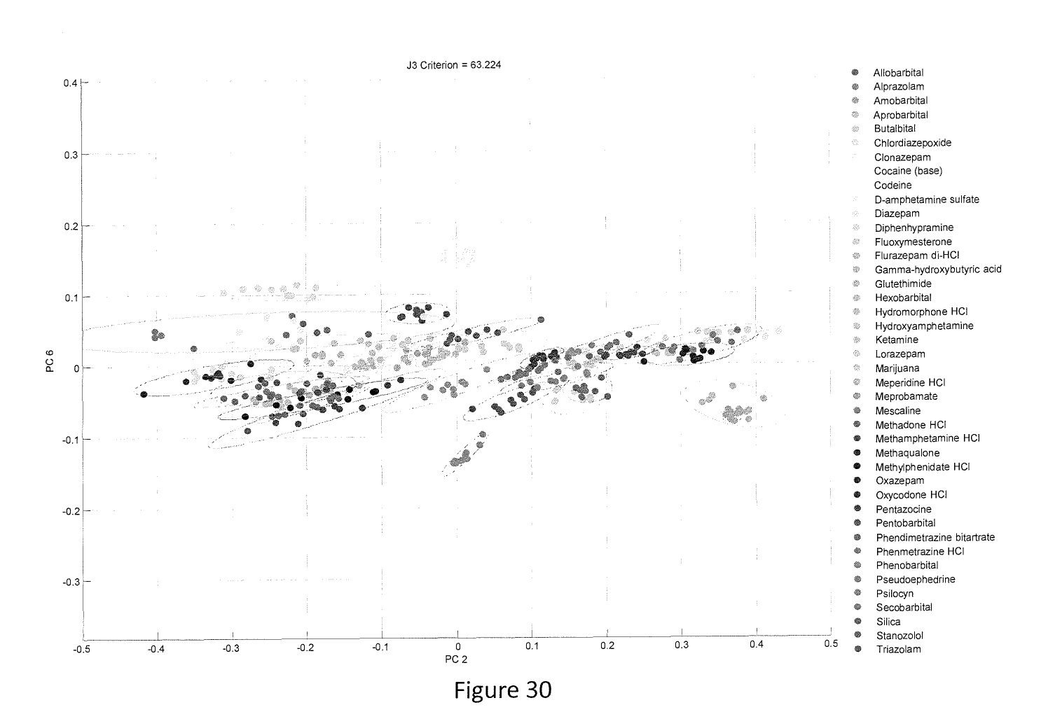

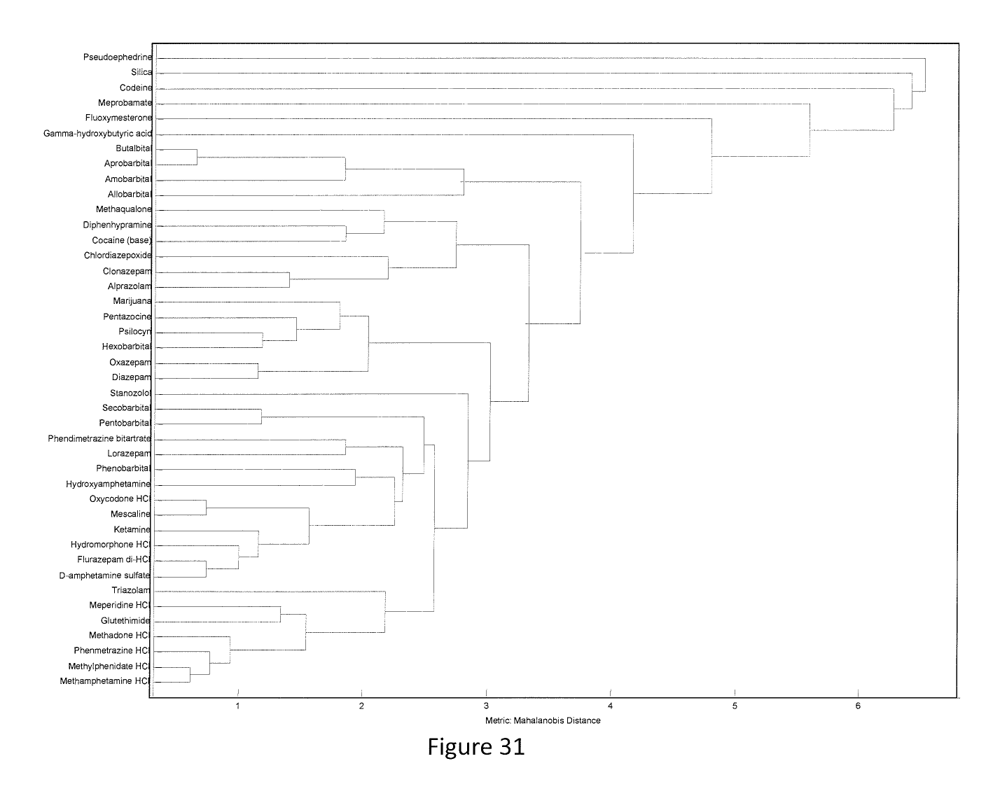

FIGS. 18-29 illustrate video images, SWIR images, and spectra associated with each material deposited in FIG. 17. The data was analyzed using a method of the present disclosure by applying PLSDA and a scatter plot showing the results of this analysis is illustrated in FIG. 30. As can be seen from FIG. 30, the drug materials can be discriminated from one another using this approach. FIG. 31 is illustrative of the application of another embodiment of a method of the present disclosure applying a MD algorithm to the data. This metric displays a similarity of an unknown sample to a known sample (such as a known drug material). The dendogram illustrates the ability to differentiate between the drug materials. Unknown materials may be associated with known drug materials based on this similarity. These results illustrate the potential for SWIR hyperspectral imaging and/or spectroscopy for detecting and/or identifying drug materials.

The present disclosure may be embodied in other specific forms without departing from the spirit or essential attributes of the disclosure. Accordingly, reference should be made to the appended claims, rather than the foregoing specification, as indicating the scope of the disclosure. Although the foregoing description is directed to the embodiments of the disclosure, it is noted that other variations and modification will be apparent to those skilled in the art, and may be made without departing from the spirit or scope of the disclosure.

What is claimed is:

1. A system for detecting drug materials comprising:
a first collection lens configured to collect a first plurality of interacted photons from a first location comprising an unknown material;
a visible imaging device configured for detecting the plurality of interacted photons and generating a visible image of the first location;
a second collection lens for focusing and locating a second location comprising at least one unknown material and collecting a second plurality of interacted photons from the second location;
a tunable filter for filtering the second plurality of interacted photons into a plurality of wavelength bands;
a spectroscopic imaging device configured to detect the second plurality of interacted photons and generate a SWIR hyperspectral image of the second location; and
at least one processor configured to analyze the SWIR hyperspectral image to associated the unknown material with at least one known material, wherein the known material comprises at least one drug.

2. The system of claim 1 wherein the second collection lens further comprises a telescope optic.

3. The system of claim 1 further comprising at least one illumination source for illuminating at least one of the first location and the second location and generating at least one of the first plurality of interacted photons and the second plurality of interacted photons.

4. The system of claim 3 wherein the illumination source further comprises at least one of: a laser light source, a broadband light source, and an ambient light source.

5. The system of claim 1 wherein the visible imaging device further comprises a RGB camera.

6. The system of claim 1 wherein the tunable filter further comprises at least one of: a Fabry Perot angle tuned filter, an acousto-optic tunable filter, a liquid crystal tunable filter, a Lyot filter, an Evans split element liquid crystal tunable filter, a Sole liquid crystal tunable filter, a fixed wavelength Fabry Perot tunable filter, an air-tuned Fabry Perot tunable filter, a mechanically-tuned Fabry Perot tunable filter, and a liquid crystal Fabry Perot tunable filter.

7. The system of claim 1 wherein the spectroscopic imaging device further comprises at least one of: an InGaAs Detector, an InSb detector, a CCD detector, an ICCD detector, and a MCT detector.

8. The system of claim 1 further comprising at least one reference data base, wherein each reference database comprises at least one reference data set, wherein each reference data set is associated with a known drug material.

9. A method for detecting drug materials comprising:
surveying a first location to thereby identify a second location comprising at least one unknown material;
collecting a plurality of interacted photons generated by the second location;
filtering the interacted photons into a plurality of wavelength bands;
detecting the plurality of interacted photons and generating at least one SWIR hyperspectral image representative of the second location; and
analyzing the SWIR hyperspectral image to associate the unknown material with a known drug material.

10. The method of claim 9 wherein analyzing the SWIR hyperspectral image further comprises comparing the SWIR hyperspectral image with at least one reference data set, wherein each reference data set is associated with a known drug material.

11. The method of claim 10 wherein the comparison is achieved by applying at least one of: a cheomemetric technique and a ratiometric technique.

12. The method of claim 9 wherein surveying the first location is further achieved by using a visible imaging device.

13. The method of claim 9 wherein the second location is selected based on at least one of size, shape, and color.

14. The method of claim 9 wherein filtering the interacted photons further comprises passing the interacted photons through at least one tunable filter.

15. A non-transitory data storage medium containing program code, which, when executed by a processor, causes the processor to:
collect a plurality of interacted photons generated by a second location;
filter the interacted photons into a plurality of wavelength bands;
detect the plurality of interacted photons and generate at least one SWIR hyperspectral image representative of the second location;
and analyze the SWIR hyperspectral image to associate the unknown material with a known drug material.

16. The non-transitory data storage medium of claim 15 wherein, when executed by a processor, further causes the processor to compare the SWIR hyperspectral image with at least one reference data set, wherein each reference data set is associated with at least one known material.

17. The non-transitory data storage medium of claim 16 wherein, when executed by a processor further causes the processor to achieve the comparison by applying at least one algorithmic technique.
